(19) United States
(12) Patent Application Publication
(10) Pub. No.: US 2018/0303725 A1
(43) Pub. Date: Oct. 25, 2018

(54) CUSTOMIZABLE DISSOLVABLE OBJECTS AND RELATED METHODS OF MANUFACTURE

(71) Applicant: Robert Nathan Alder, Greenacres, FL (US)

(72) Inventor: Robert Nathan Alder, Greenacres, FL (US)

(21) Appl. No.: 15/959,049

(22) Filed: Apr. 20, 2018

Related U.S. Application Data
(60) Provisional application No. 62/488,481, filed on Apr. 21, 2017.
(60) Provisional application No. 62/522,854, filed on Jun. 21, 2017.

(51) Int. Cl. A61K 8/02; A61Q 19/10

(52) U.S. Cl. CPC A61K 8/022; A61Q 19/10; A61K 2800/65; A61K 2800/222; A61K 2800/10

(57) ABSTRACT

The present disclosure is directed to dissolvable objects that are configurable. The dissolvable objects are dissolvable in a solvent, such as water in a bath tub. The dissolvable objects are further configured to receive a seletable additive, which can be an insertion of another object and/or liquid. The present disclosure is also directed to methods of manufacturing dissolvable objects that are configurable.

### CROSS-REFERENCE TO RELATED APPLICATIONS

This application claims the benefit under 35 U.S.C. § 119(e) of U.S. Provisional Application No. 62/488,481, filed on Apr. 21, 2017 and titled “CUSTOMIZATION OF DISSOLVABLE OBJECTS,” and of U.S. Provisional Application No. 62/522,854 filed on Jun. 21, 2017 and titled “CUSTOMIZABLE DISSOLVABLE OBJECTS AND RELATED METHODS OF MANUFACTURE,” which are hereby incorporated by reference in their entirety.

### FIELD OF THE INVENTION

The present disclosure relates generally to dissolvable objects that can be put in an agent, liquid, or environment, such as a bath, that causes that object to dissolve and release fragrances and/or oils or other dermatologic or skin-care substances.

### BACKGROUND

Presently, effervescent dissolvable objects (e.g., a bath bomb) include a mixture of reactive ingredients that are combined and formed into a sphere (or other three-dimensional shape). In a formed state, these presently available dissolvable objects are dry and do not react until placed in a liquid which begins to dissolve the sphere and release the contents of the sphere.

The presently available dissolvable objects are distributed premade in a formed state and are not alterable, and cannot be modified or otherwise configured or customized. Presently available dissolvable objects generally cannot be opened, and are not capable of receiving (or of having anything inserted) inside to customize the dissolvable object. For example, the fragrances and/or oils of the dissolvable object cannot be modified or otherwise customized.

Presently available dissolvable objects in the formed (e.g., pre-made, dry) state have a short shelf life of a couple weeks as the fragrances and other ingredients evaporate from the dissolvable object.

Existing manufacturing methods to form dissolvable objects don't allow for any insertion of external items. There would also be a fear of the object collapsing on itself as normal manufacturing is to press two half-sphere molds together firmly to pack and bind the ingredients and contents and to collapse cavities.

The foregoing challenges relating to lack of customizability and longevity of presently available dissolvable objects can be addressed by the disclosed embodiment.

### BRIEF DESCRIPTION OF THE DRAWINGS

FIG. 1A is a cross-sectional view of a dissolvable object, according to one embodiment of the present disclosure, that includes a channel.

FIG. 1B is a cross-sectional view of the dissolvable object of FIG. 1A, with a customization receptacle or capsule inserted into the channel.

FIG. 2 is a cross-sectional view of a dissolvable object, according to another embodiment of the present disclosure, including a channel that is drilled to allow for a customization receptacle or capsule to be inserted inside the object.

FIG. 3 is a cross-sectional view of a dissolvable object, according to another embodiment of the present disclosure, formed in two halves that, when combined, form a cavity and an external item can be inserted in the cavity prior to combining the two halves.

FIG. 4 is a cross-sectional view of a dissolvable object, according to another embodiment of the present disclosure, wrapped in an air-tight outer layer.

FIG. 5A is a cross-sectional view illustrating one step of a method of manufacturing a dissolvable object, according to one embodiment of the present disclosure.

FIG. 5B is a cross-sectional view illustrating another step of a method of manufacturing a dissolvable object, according to the embodiment depicted in FIG. 5A of the present disclosure.

FIG. 5C is a cross-sectional view illustrating yet another step of a method of manufacturing a dissolvable object, according to the embodiment depicted in FIG. 5A of the present disclosure.

FIG. 6A is a cross-sectional view illustrating one step of a method of manufacturing a dissolvable object, according to another embodiment of the present disclosure.

FIG. 6B is a cross-sectional view illustrating another step of a method of manufacturing a dissolvable object, according to the embodiment depicted in FIG. 6A of the present disclosure.

### DETAILED DESCRIPTION

In the following detailed description, reference is made to the accompanying drawings, which form a part hereof, and in which is shown by way of illustration specific embodiments in which the present disclosure may be practiced. These embodiments are described in sufficient detail to enable those of ordinary skill in the art to practice the disclosure made herein. It should be understood, however, that the detailed description and the specific examples, while indicating examples of embodiments of the disclosure, are given by way of illustration only, and not by way of limitation. From the disclosure, various substitutions, modifications, additions, rearrangements, or combinations thereof within the scope of the disclosure may be made and will become apparent to those of ordinary skill in the art.

In one embodiment of the present disclosure, one or more channels is molded or cut into a dissolvable object so as to allow an external item or liquid to be inserted inside a main object body of the dissolvable object.

In another embodiment of the present disclosure, a cavity can be created within a main object body of a dissolvable object to allow a space for an external item to rest and be trapped or retained until the outer main object body dissolves.

In another embodiment of the present disclosure, an access channel can be formed within a main object body to insert an external object. The access channel can be left open or closed with a plug or door.

In another embodiment of the present disclosure, a liquid or powder is contained within a dissolvable object, and the liquid or powder is released and can mix with other contents of the dissolvable object.

In another embodiment of the present disclosure, a liquid or powder is contained within a dissolvable object and releases or mixes with other contents, including the main object body (i.e. a bath bomb that is wrapped in a dissolvable wrapper/shell/capsule/receptacle).

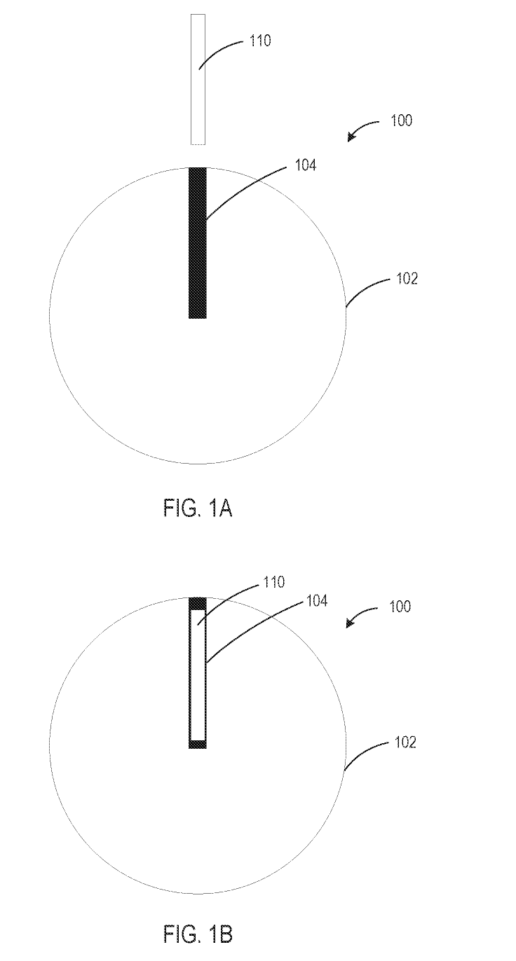

FIG. 1A is a dissolvable object (e.g., a bath enhancing article) 100, according to one embodiment of the present disclosure. The dissolvable object includes an object body 102 and a channel 104 formed in the object body 102. The dissolvable object of FIG. 1A further includes one or more customization capsules 110 (or receptacles, liquids, or other selectable additives) that can be inserted into and received within the channel 104. The channel 104 may be formed in the object body 102 of the dissolvable object 100 during a manufacturing process so that when the object body 102 dries and/or cures to a formed state the shape of a channel and/or cavity will remain in the object body 102. In other embodiments, the channel 104 is formed after the object body 102 dries and or cures. The channel 104 may pass part or all the way through the object body 102.

The customization capsule 110 may be formed of a dissolvable film, wrapper, shell, or the like, or other material that may similarly dissolve in a dissolving liquid or solvent that dissolves the object body 102. The customization capsules 110 may include one or more customization substances or additives in a liquid or powder form, such as fragrances, vitamins, nutrients, antioxidants, stimulants, pheromones, and/or oils or other dermatologic or skin-care substances or substances that can be absorbed by the skin into the body. These customization substances are secured within the capsule retained from evaporation or other dissipation from passage of time. Moreover, the customization capsule(s) 110 or liquid enables a user of the dissolvable object to select a combination of customization substances or additives according to a present preference or desire prior to placement of the dissolvable object 100 into bath water.

FIG. 1B is the dissolvable object 100 of FIG. 1A, with a customization capsule 110 inserted into the channel 104. The customization capsule 110 may be secured within the channel 104 by friction (e.g., a friction fit), a plug, a door, or other securement mechanism. When placed in a liquid (e.g., water) or other solvent able to dissolve the object body 102, the dissolvable object 100 gradually dissolves, initially by dissolution of the object body 102 and eventually including dissolution of the customization capsule 110. The eventual dissolution of the customization capsule 110 releases a desired combination of customization substances into the solution.

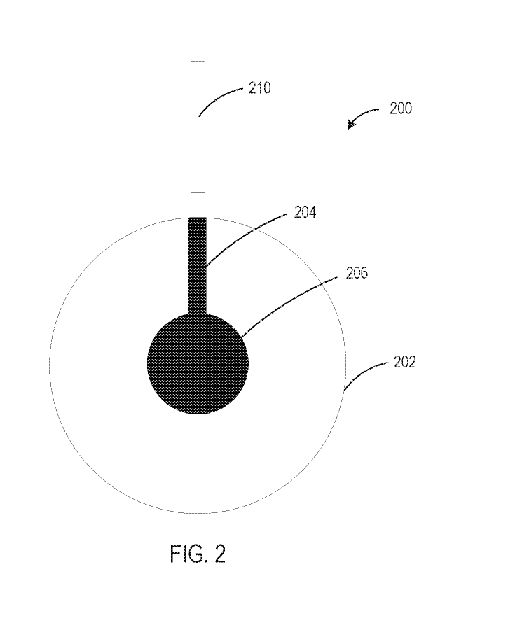

FIG. 2 is a dissolvable object 200, according to another embodiment of the present disclosure. The dissolvable object 200 includes a cavity 204 that may be drilled or otherwise to provide such capabilities to allow for an external object 210 to be inserted inside a cavity 206 within an object body 202 of the dissolvable object 200. The external object 210 may be a customization capsule, one or more capsules of essential oils, toys, flowers, pheromones, vitamins, antioxidants, fragrances, nutrients, stimulants, other chemicals, or other dermatologic or skin-care substances or substances that can be absorbed by the skin into the body. The external object 210 may be inserted inside the hole and may be lodged inside the channel 204, or rest inside the cavity 206 to prevent escape.

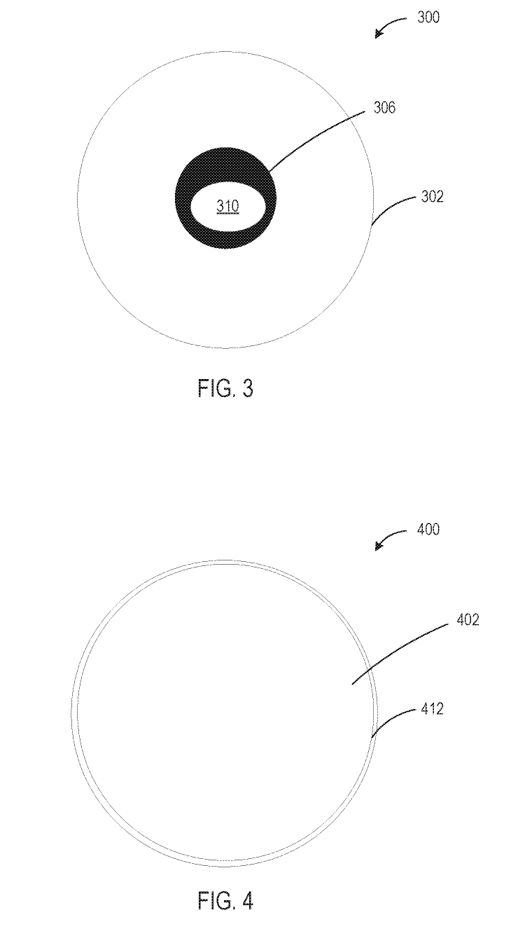

FIG. 3 is a cross-sectional view of a dissolvable object 300, according to another embodiment of the present disclosure. The dissolvable object 300 includes an object body 302 that is formed in two halves that, when combined, form a cavity 306. An external item 310 can be inserted in the cavity 306 prior to combining the two halves of the object body 302. The external item 310 may be a capsule of oil or vitamins etc.

In still another embodiment, the dissolvable object 300 may be simply formed around an external item 310, such as a capsule. The capsule may include oil or vitamins, etc., but the capsule or other protective coating around the oil or vitamins, etc. may prolong a shelf life of the dissolvable object 300, albeit without allowing customization after formation.

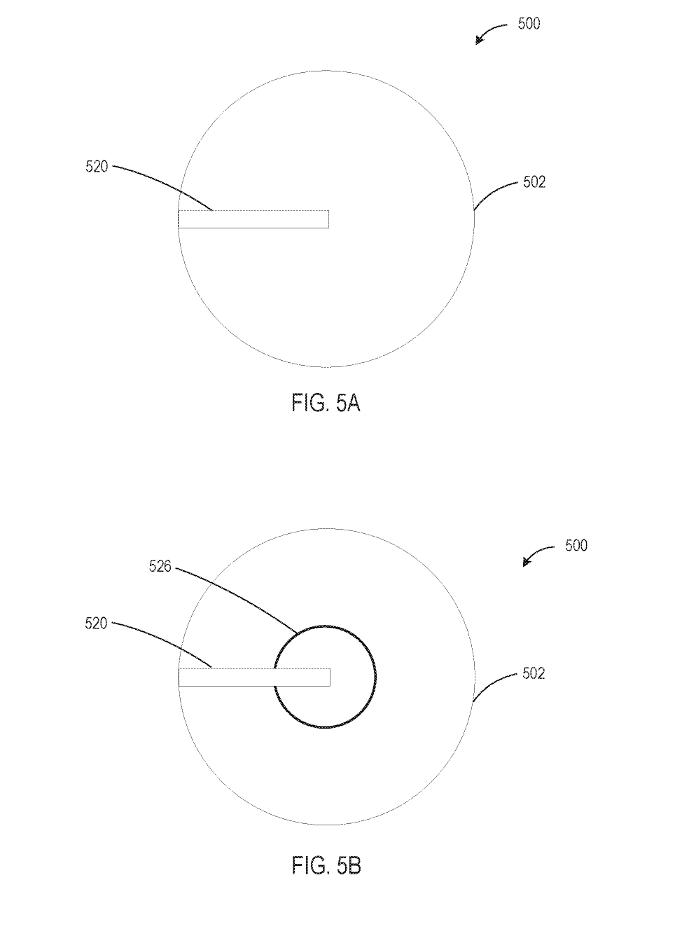

FIG. 4 is a cross-sectional view of a dissolvable object 400, according to another embodiment of the present disclosure. The dissolvable object 400 includes a main body 402 and a dissolvable outer layer 412 that encloses (e.g., wraps, seals, covers) the main body 402 to preserve any fragrances that may be included in the main body. Stated differently, a main body 402 (e.g., formed of an effervescent powder) may be sealed in a dissolvable air tight packaging to prolong a shelf life of the main body 402.

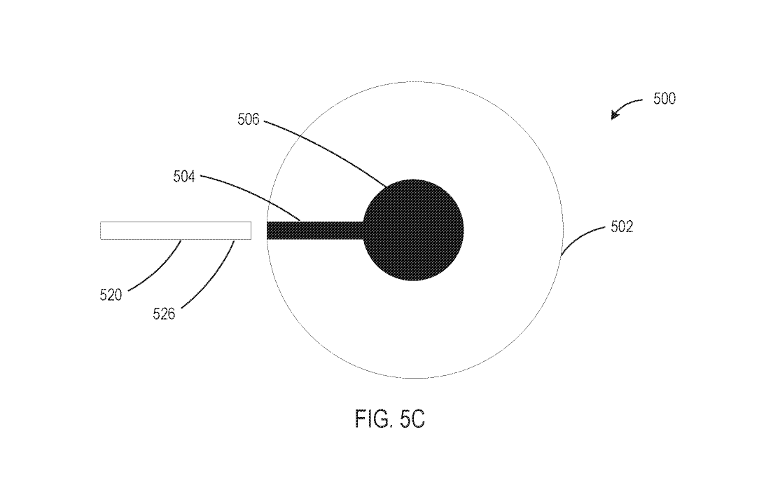

FIG. 5A is a cross-sectional view illustrating one step of a method of manufacturing a dissolvable object 500, according to one embodiment of the present disclosure. The dissolvable object 500 includes a main body 502 that may be penetrated by an external device 520 configured to penetrate into the main body 502 of the dissolvable object 500. For example, the external device 520 may remove a portion of the material comprising the main body 502 of the dissolvable object 500. A drilling action of the external device 520 may remove material of the main body 502 to form a channel 504. In another embodiment, the external device 520 may be forcefully inserted into the main body 502, such as after the manner of taking a core sample, thereby displacing the material of the main body 502.

FIG. 5B is a cross-sectional view illustrating another step of the method of manufacturing a dissolvable object 500 of FIG. 5A. The dissolvable object 500 includes a main body 502 that may have been penetrated by an external device 520 configured to be inserted into the main body 502 of the dissolvable object 500. The external device 520 of FIG. 5B includes a cavity formation tip 526 that may be configured to displace or remove additional material within the main body 502 at a distal end of the external device 520 after the device has been inserted. For example, the cavity formation tip 526 may include an expandable distal end configured to expand to displace additional material within the main body 502 when the expandable distal end is pressurized. In one embodiment, a fluid (i.e., hydraulic forces) may be used to pressurize the expandable distal end of the cavity formation tip 526 thereby causing a portion of the material within the main body 502 to be displaced. Alternatively, in other embodiments, gas (i.e., pneumatic forces) may be used to pressurize the expandable distal end of the cavity formation tip 526 thereby causing a portion of the material within the main body 502 to be displaced.

FIG. 5C is a cross-sectional view illustrating yet another step of the method of manufacturing a dissolvable object 500, of FIG. 5A. The dissolvable object 500 includes a main body 502 that may have been penetrated by an external device 520 configured to be inserted into the main body 502 of the dissolvable object 500 and displace or remove material at an expandable distal end of the external device 520. After the material within the main body 502 has been removed or displaced, the expandable distal end of the external device 520 may be retracted from a cavity 506 formed by the external object 520 (e.g., with the expandable distal end of the cavity formation tip 526) within the main body 502 of the dissolvable object 500. As the external device 520 is also removed, a channel 504 remains formed within the main body 502 of the dissolvable object 500.

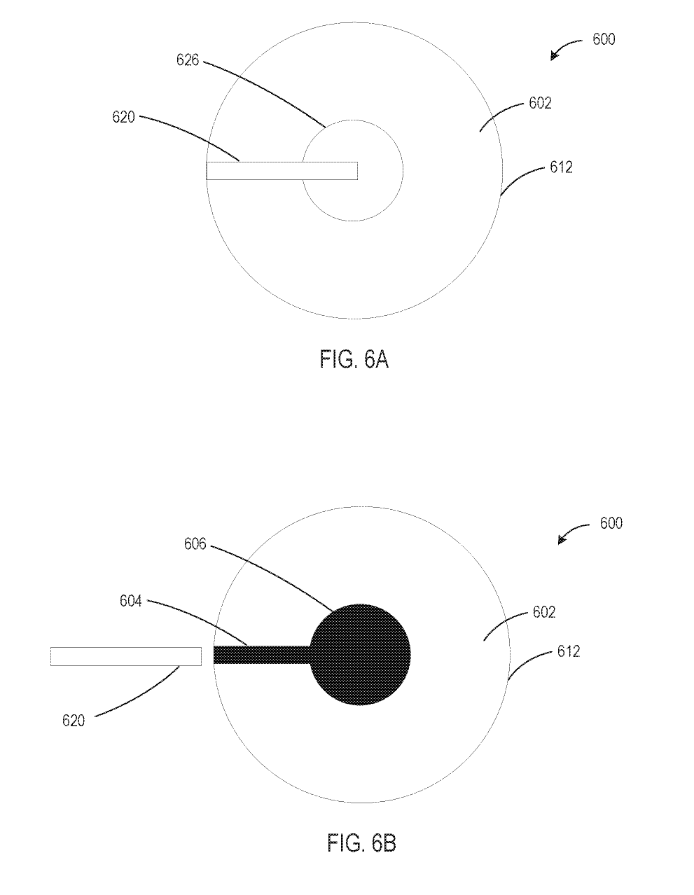

FIG. 6A is a cross-sectional view illustrating one step of a method of manufacturing a dissolvable object 600, according to another embodiment of the present disclosure. The dissolvable object 600 may be formed from a mold 612. The mold 612 may be configured to partially encase an external device 620 within the main body 602 of the dissolvable object 600 within the mold 612. The external device 620, partially encased within the mold 612, includes a cavity formation tip 626 that may be configured to inhibit material placed within the mold 612 from occupying a portion of the volume within the mold 612. When material is placed within the mold 612, the expandable distal end of the external device 620 may be expanded and thereby inhibit material placed within the mold 612 from occupying the entirety of the volume within the mold 612. For example, the cavity formation tip 626 may include an expandable distal end configured to expand to occupy a portion of the volume within the mold 612 when the expandable distal end is pressurized. In one embodiment, a fluid (i.e., hydraulic forces) may be used to expand the expandable distal end of the cavity formation tip 626 thereby occupying a portion of the volume within the mold 612. Alternatively, in other embodiments, gas (i.e., pneumatic forces) may be used to pressurize the expandable distal end of the cavity formation tip 626 thereby occupying a portion of the volume within the mold 612 when the expandable distal end of the cavity formation tip 626 is pressurized.

FIG. 6B is a cross-sectional view illustrating another step of the method of manufacturing a dissolvable object 600, of FIG. 6A. The mold 612, partially encasing an external device 620 having a cavity formation tip 626, may be filled with a material to create a dissolvable object 600. After the material placed within the mold 612 has hardened (e.g., compressed, cured, set, etc.), the expandable distal end of the cavity formation tip 626 may be retracted or rescinded as to allow removal of the external device 620 from the main body 602 of the dissolvable object 600 and the mold 612 and leave a cavity 606 and channel 604 within the mold 612 free of material.

### EXAMPLES

A dissolvable object configured to dissolve in a solvent, the dissolvable object configured to receive an insertion of another object or liquid inside.

A bath enhancing article comprising: a body; and a selectable additive configured as any one of a receptacle, a liquid, and a combination thereof; wherein the body is configured to receive the selectable additive prior to placing the body into bath water.

An object configured to dissolve in a substance, liquid, or environment, the object comprising a hole cut part or all the way through the object.

An object configured to dissolve in a substance, liquid, or environment, the object comprising a cavity inside the object.

Any liquid or powder contained within a dissolvable object that releases or mixes with other contents

Any liquid or powder contained within a dissolvable object that releases or mixes with other contents, including the main object (i.e. a bath bomb that is wrapped in a dissolvable wrapper/shell/capsule.

A method for forming a dissolvable object, comprising: placing a rod or channel forming device through the external surface of the object until reaching a desired inner position; expanding a distal end of the rod or channel forming device, thereby forcing material within the object to be displaced; retracting or rescinding the distal end of the rod or channel forming device; extracting or removing the rod or channel forming device from the object leaving the object with a cavity inside.

A method for manufacturing a dissolvable object may include: placing a rod or channel forming device within a mold at a desired inner position; expanding a distal end of the rod or channel forming device within the mold; depositing material within the mold; retracting or rescinding the distal end of the rod or channel forming device; extracting or removing the rod or channel forming device from the mold leaving the a cavity within the deposited material.

A method for forming a dissolvable object may include: drilling a hole through an external surface of the object until reaching a desired inner position; expanding a distal end of the drill while drilling thereby removing material within the object; retracting or rescinding the distal end of the drill; extracting or removing the drill from the object leaving the object with a cavity inside.

1. A dissolvable object comprising:
a dissolvable object body; and
a channel disposed within the dissolvable object body;
wherein the dissolvable object body is configured to dissolve in a solvent and to receive an insertion of any one of a receptacle, a liquid, and a combination thereof within the channel.

2. The dissolvable object of claim 1, wherein the dissolvable object body comprises an effervescent powder.

3. The dissolvable object of claim 2, wherein the receptacle comprises any one of a fragrance, a vitamin, a nutrient, an oil, a dermatologic substance, an antioxidant, a stimulant, and any combination thereof to mix or release with the effervescent powder into the solvent.

4. The dissolvable object of claim 2, wherein the liquid comprises any one of a fragrance, a vitamin, a nutrient, an oil, a dermatologic substance, an antioxidant, a stimulant, and any combination thereof configured to mix or release with the effervescent powder into the solvent.

5. The dissolvable object of claim 1, wherein the channel extends from an exterior surface of the dissolvable object body into at least a portion of the dissolvable object body.

6. The dissolvable object of claim 1, further comprising a cavity disposed adjacent a distal end of and in fluid communication with the channel.

7. The dissolvable object of claim 1, wherein the receptacle forms a capsule comprising a dissolvable shell.

8. The dissolvable object of claim 6, wherein the receptacle is configure to be secured within one of the channel, the cavity, and a combination thereof.

9. The dissolvable object of claim 1, wherein the dissolvable body comprises a dissolvable shell.

10. A bath enhancing article comprising:
a body that is dissolvable in bath water, the body defining a channel therein; and
a selectable additive configured as any one of a receptacle, a liquid, and a combination thereof;
wherein the body is configured to receive the selectable additive within the channel prior to placing the body into bath water.

11. The bath enhancing article of claim 10, wherein the body comprises an effervescent powder.

12. The bath enhancing article of claim 11, wherein the receptacle comprises any one of a fragrance, a vitamin, a nutrient, an oil, a dermatologic substance, and any combination thereof configured to mix or release with the effervescent powder into the bath water.

13. The bath enhancing article of claim 11, wherein the liquid comprises any one of a fragrance, a vitamin, a nutrient, an oil, a dermatologic substance, and any combination thereof configured to mix or release with the effervescent powder into the bath water.

14. The bath enhancing article of claim 11, further comprising a cavity configured to receive any one of the receptacle, the liquid, and any combination thereof.

15. A method of manufacturing a dissolvable object, comprising:
placing a channel forming device through an external surface of the dissolvable object to a desired inner position; and
extracting the channel forming device from the dissolvable object, wherein the dissolvable object comprises a channel after extraction of the channel forming device.

16. The method of claim 15, further comprising:
expanding a distal end of the channel forming device, wherein material within the dissolvable object is radially displaced to form a cavity; and
collapsing the distal end of the channel forming device.

17. The method of claim 15, further comprising:
placing the channel forming device within a mold at a desired inner position;
depositing a dissolvable material within the mold to form the dissolvable object; and
removing the channel forming device from the mold and the dissolvable object to form the channel.

18. The method of claim 17, further comprising:
expanding a distal end of the channel forming device within the mold and the dissolvable object to radially displace dissolvable material to form a cavity; and
collapsing the distal end of the channel forming device.

19. The method of claim 15, further comprising:
drilling a hole through an external surface of the dissolvable object to form the channel; and
removing the drill from the dissolvable object.

20. The method of claim 19, further comprising:
expanding a distal end of the drill while drilling to remove material within the dissolvable object to form a cavity; and
retracting the distal end of the drill.
